(19) United States
(12) Patent Application Publication
(10) Pub. No.: US 2012/0264825 A1
(43) Pub. Date: Oct. 18, 2012

(54) Amino Acid Perhydrates, Process for Their Preparation and Uses thereof

(76) Inventors: Ovadia Lev, Jerusalem (IL); Petr Prikhodchenko, Givat-Ram (IL)

(21) Appl. No.: 13/319,218

(22) PCT Filed: May 6, 2010
(86) PCT No.: PCT/IL10/00370

§ 371 Date: Jun. 25, 2012

Related U.S. Application Data
(60) Provisional application No. 61/176,203, filed on May 7, 2009.

(51) Int. Cl. C01B 15/017; A61K 8/44; C07C 229/34; C07C 229/22; C07C 229/08

(52) U.S. Cl. 514/561; 562/567; 562/553; 562/443; 562/445; 562/570; 562/576; 423/584

(57) ABSTRACT

The invention provides compounds which are -amino acid hydrogen peroxide solvates, wherein the side chain of the -amino acid has no basic nitrogen. A process for preparing the compounds and uses thereof are also described.

### FIELD OF THE INVENTION

Hydrogen peroxide is an environmentally friendly oxidizer that is routinely used in fine chemical synthesis, catalysis, and the electronic industry. Hydrogen peroxide is readily available in the form of aqueous solutions. For many applications, however, hydrogen peroxide is needed in an anhydrous form or in solid state.

One approach to preparing anhydrous solutions of hydrogen peroxide involves the use of adducts (or complexes) of hydrogen peroxide with an organic compound. Upon dissolution in a suitable organic solvent, the adduct releases the hydrogen peroxide into the solution. Examples of such adducts include urea peroxide and the 1:2 complex of 1,4-diazabicyclo[2.2.2]octane (DABCO):hydrogen peroxide. Unfortunately, the organic components of the adducts mentioned above remain in the hydrogen peroxide product solution, and may interfere with the contemplated catalytic and synthetic utilities of the anhydrous hydrogen peroxide solution.

The preparation of histidine-hydrogen peroxide adduct was described by Dirscherel et al. [Kurze Originalmitteilungen, p. 552 (1954)] and in U.S. Pat. No. 5,122,354. The molecular structure of the adduct, resolved by X-rays analysis reported in U.S. Pat. No. 5,122,354, appears to involve the formation of hydrogen bonding between the hydrogen peroxide and the nitrogen atom of the imidazole ring of histidine.

### SUMMARY OF THE INVENTION

It has now been found that α-amino acids, which have no basic nitrogen in their side chain, can be recovered from aqueous hydrogen peroxide solutions in the form α-amino acid hydrogen peroxide solvates. A basic nitrogen is a nitrogen which can accept proton, e.g., primary, secondary or tertiary amine.

The α-amino acid hydrogen peroxide solvate of the invention may be either crystalline or amorphous. Thus, the term “solvate” shall refer herein not only to a true solvate of the α-amino acid, in which the hydrogen peroxide molecule is held within the crystal lattice, but also to the pseudomorph of the α-amino acid in an amorphous form. It has been found, however, that the solvates of the invention generally exist in a crystalline form. Hereinafter, the terms “amino acid perhydrates” and “perhydrates” are sometimes used to indicate the α-amino acid hydrogen peroxide solvate of the invention.

The present invention therefore primarily relates to α-amino acid hydrogen peroxide solvate, wherein the side chain of the α-amino acid has no basic nitrogen.

### DETAILED DESCRIPTION OF THE INVENTION

One class of α-Amino acids prepared and isolated in the form of perhydrates according to the invention includes nonpolar α-amino acids. The nonpolar side chain of said α-amino acids may consist of hydrogen atom or hydrocarbon groups, e.g., C1-C5 alkyl or aryl groups. α-Amino acids having nonpolar side chains, which can be crystallized in the form of hydrogen peroxide solvates according to the invention, are, for example, valine, leucine, isoleucine, norleucine, 2-Aminobutyric acid and phenylalanine.

Another class of α-amino acids that were crystallized in the form of perhydrates according to the invention includes polar α-amino acids, having one or more polar groups in their side chain. For example, α-amino acids having hydroxyl functionality in their side chain, e.g., serine, threonine and tyrosine, were prepared and isolated as perhydrates.

It should be understood that the term “α-amino acids” shall refer herein not only to the group of α-amino acids of which protein are composed (called proteinogenic or standard amino acids), but also to non-standard α-amino acids, which in the context of the present invention are derivatives of standard α-amino acids in which a slight structural modification is present (in comparison to the side chain of a proteinogenic α-amino acid). For example, a non-standard α-amino acid which may form hydrogen peroxide solvate according to the invention may have a side chain consisting of a structural isomer or homolog of a “standard” linear or branched alkyl chain, wherein the homolog differs in length from the “standard” linear or branched alkyl chain by one or two methylene groups. Indeed, two of the examples listed above (norleucine and 2-Aminobutyric acid) represent non-standard α-amino acids. A non-standard α-amino acid can also be hydroxy-substituted derivative of a proteinogenic α-amino acid.

The α-amino acid perhydrates are prepared by dissolving the α-amino acid in a solution of hydrogen peroxide, e.g., in an aqueous solution of hydrogen peroxide, causing the product to precipitate from the solution and separating the solid formed, as described in more detail below. The α-amino acid hydrogen peroxide solvate of the invention is preferably recovered in an anhydrous form, but it is also possible to prepare a mixed solvate, namely, α-amino acid perhydrate/hydrate. Thus, the compounds according to the invention may comprise solely α-amino acid and hydrogen peroxide molecules, or alternatively may also comprise water molecules, and are hence conveniently represented by the following formula:

α-amino acid.x(H₂O₂).y(H₂O)

wherein the side chain of said α-amino acid is selected from the group consisting of:
(i) the side chains of proteinogenic α-amino acids (other than the basic α-amino acids, namely, histidine, arginine and lysine)
(ii) structural isomers or homologs of proteinogenic α-amino acid side chains, when said side chains consist of alkyl groups; and
(iii) hydroxy-substituted derivatives of proteinogenic α-amino acid side chains (for example, when said side chains consist of arylic groups (such as benzyl));
the coefficient x in the range between ⅓ to 4, for example between ½ to 2; in particular, x equals 1, 1.5 or 2 (corresponding to the mono-, sesqui- and di-hydrogen peroxide solvates, respectively); and
the coefficient y is in the range between 0 and 1, preferably between 0 and 0.5.

It should be noted that for certain utilities which are described in detail hereinafter, it is preferred to prepare the α-amino acid hydrogen peroxide solvate in an anhydrous form, or at least in a substantially anhydrous form. By the term “substantially anhydrous form” is meant a mixed α-amino acid perhydrate/hydrate wherein the amount of water of hydration contained in the crystalline structure of the mixed solvate is minimized, such that the ratio y:x is in the range between 0.01 and 0.1. In general, it has been observed that in the mixed α-amino acid perhydrate/hydrate of the invention, the sum of the coefficients x and y is 1, 1.5 or 2.

It may be appreciated that some of the α-amino acid perhydrates provided by the invention have high hydrogen peroxide content, of not less than 18% by weight, for example, between 18 and 45% by weight. Furthermore, some of the compounds provided by the invention, wherein x is greater than 1 (e.g., the sesqui- and di-hydrogen peroxide solvates) may be regarded as particularly rich organoperhydrate.

Especially preferred compounds of the invention are the crystalline α-amino acid hydrogen peroxide solvates and mixed hydrogen peroxide/water solvates listed below:
L-Serine hydrogen peroxide solvate [C₃H₇NO₃.H₂O₂)]
L-Tyrosine dihydrogen peroxide solvate [C₉H₁₁NO₃.2(H₂O₂)]
Glycine hydrogen peroxide sesquisolvate [C₂H₅NO₂.1.5(H₂O₂)]
L-Phenylalanine hydrogen peroxide solvate hemihydrate [C₉H₁₁NO₂.H₂O₂.0.5(H₂O)]
L-Serine 0.91 hydrogen peroxide solvate 0.09 hydrate [C₃H₇NO₃.0.91(H₂O₂).0.09(H₂O)]
L-Phenylalanine 1.43 hydrogen peroxide solvate 0.07 hydrate [C₉H₁₁NO₂.1.43(H₂O₂).0.07(H₂O)]
L-Isoleucine hydrogen peroxide solvate hemihydrate [C₆H₁₃NO₂.H₂O₂.0.5(H₂O)]
L-Threonine hydrogen peroxide solvate [C₄H₉NO₃.H₂O₂]
L-Norleucine hydrogen peroxide sesquisolvate [C₆H₁₃NO₂.1.5H₂O₂]
2-Aminobutyric acid hydrogen peroxide sesquisolvate [C₄H₉NO₂.1.5(H₂O₂)]
L-Valine hydrogen peroxide solvate.

The invention further provides a process for producing α-amino acid hydrogen peroxide solvate, comprising the steps of (a) providing a solution of hydrogen peroxide, (b) contacting the solution with an α-amino acid and (c) precipitating the α-amino acid hydrogen peroxide solvate and separating the same from the solution.

Regarding step (a), the hydrogen peroxide solution used is preferably an aqueous solution with a concentration of not less than 25% (w/w), more preferably not less than 50% (w/w). The concentration of the aqueous hydrogen peroxide solution is preferably between 50 and 98% by weight. The aqueous hydrogen peroxide solution may comprise a co-solvent, i.e. water-miscible organic solvent. Alternatively, the hydrogen peroxide used in the process of the invention is in the form of anhydrous reagent. However, in general it is most convenient to use aqueous hydrogen peroxide solutions, despite the fact that the water molecule is a potential competitor of the hydrogen peroxide molecule in the solvate formation process. It has been observed that an increase of the concentration of the hydrogen peroxide solute in the aqueous solution employed minimizes the amount of water of hydration in the product formed. Thus, if anhydrous α-amino acid hydrogen peroxide solvates are contemplated, then it is preferable to employ an aqueous hydrogen peroxide solution having concentration of not less than 70% by weight. Commonly used stabilizers, such as phosphate-based preservatives, may be present in the hydrogen peroxide solution.

Regarding step (b), the α-amino acid, provided either in a solid (powder or granular) form or as a pre-formed solution, is added to the hydrogen peroxide solution and is allowed to dissolve therein. The concentration of the α-amino acid in the hydrogen peroxide solution is preferably about the saturation limit, which is generally not less than few grams per 1 kg H₂O₂ solution.

Regarding step (c), the precipitation of the product is accomplished by techniques known in the art, e.g., by maintaining the solution obtained under temperature and for a duration sufficient to form the α-amino acid perhydrate precipitate. More specifically, the crystallization of the product is induced by cooling the solution to a temperature below 10° C., e.g., to a temperature in the range between −20 and 5° C., or by concentrating the solution, or even through the use of an antisolvent, whereby a solid is caused to precipitate. However, the preferred isolation method involves cooling the solution to induce the crystallization of the product. The cooled solution is kept at the selected temperature range for a period of time between few minutes an up to several days. The solution may be allowed to stand under said conditions even for a longer period, in order to improve the yield, and additional crops may be subsequently recovered from the mother liquor following the separation of the first crop, if desired. Optionally, a surfactant (e.g. sodium diisooctyl sulfosuccinate) may be added to the solution before step (c) to affect the size of the crystals formed.

As the concentration of hydrogen peroxide in the solution changes during the precipitation step, it may be desirable to add concentrated hydrogen peroxide to the solution or to separate the precipitate from the solution before precipitation is completed, i.e. when the solution is still super saturated with the solvate precursors.

The precipitate thus formed in the solution may be recovered using conventional solid/liquid separation methods such as filtration, centrifugation and decantation. The crystals collected may be optionally washed and dried under careful conditions in order to maintain the solvated form.

The preferred compounds of the invention are crystalline, as determined by methods such as single crystal X-ray analysis and Fourier transform infrared spectroscopy (FTIR). The crystalline characteristics of the preferred compounds of the inventions are described in the Examples below by crystallographic parameters (unit cell dimensions, space group). The single crystal X-ray analysis suggests that all ‘active’ hydrogen atoms (amino, hydroxy and hydroperoxy) are engaged in hydrogen bonding, with the hydrogen peroxide molecules acting both as donor and acceptor of hydrogen bonds, forming from 3 to 5 intermolecular hydrogen bonds.

The compounds of the invention are stable under storage. However, it is generally preferred not to expose the compounds to humid environment. The compounds may be kept, for example, in closed vessels in a 4° C. refrigerator. Some of the crystalline α-amino acid hydrogen peroxide solvates can be stored in a closed vessel at room temperature with no loss the oxidation capacity, as indicated by permanganate titration. In this regard, the anhydrous L-Serine hydrogen peroxide solvate was found to be especially stable, and maintained constant hydrogen peroxide concentration for several months at room temperature.

The compounds of the invention also exhibit high thermal stability. As opposed to urea hydrogen peroxide, which undergoes decomposition upon melting, the compounds of the invention do not exhibit concurrent melting and decomposition processes, as indicated by Differential scanning calorimetry (DSC). The preferred compounds of the invention (in particular, serine hydrogen peroxide solvate and glycine hydrogen peroxide sesquisolvate) are characterzied in that their DSC curves exhibit a melting endotherm followed by a distinct exothermic peak (the latter indicates the decomposition of the compound). The temperature difference between the two thermal events may be around 5 to 15 degrees.

Thus, the preferred compounds of the invention do not decompose upon melting, and exist also in a liquid (molten) state. Thermogravimetry analysis (TG) was consistent with observed DSC behavior, showing that the melting process was not accompanied by weight loss. Indeed, the phase transition of the compounds of the invention has been found to be reversible: the molten compounds solidify to form the crystalline solvates, as was verified visually and by the DSC study.

In view of the fact that the temperature difference between the melting and decomposition of the preferred compounds of the invention may be around 5 to 15 degrees, it is possible to use the α-amino acid hydrogen peroxide solvates in a liquid (molten) form. The thermal profile of the compounds of the invention thus offers a considerable advantage from the viewpoint of formulation processes, where various ingredients need to be thoroughly mixed in a liquid form to form homogeneous mixture, which is then allowed to solidify.

The α-amino acid hydrogen peroxide solvates of the invention are stable and easily handled reagents and are hence suitable for use in virtually all standard applications of hydrogen peroxide and of the commercially available urea-hydrogen peroxide adduct. The compounds of the invention may be used as such, or may be combined with one or more additives to form a composition tailored for the intended application. It should be noted that natural α-amino acids are approved food ingredients.

Accordingly, a composition comprising one or more α-amino acid hydrogen peroxide solvates and at least one carrier forms another aspect of the invention. The carrier may vary in accordance with the intended use, and may be solid, semi-solid or liquid. For example, for therapeutic or cosmetic compositions the carrier may be a therapeutically or cosmetically acceptable carrier, respectively, such as Vaseline, glycerol and other viscous oils.

The concentration of the α-amino acid hydrogen peroxide solvates in the composition of the invention varies in a broad range, e.g., between 0.1 and 70% (by weight, relative to the total weight of the composition). The composition is prepared by conventional methods, e.g., by blending the various ingredients to form a homogeneous mass. As illustrated in the examples below, the compound of the invention was easily formulated together with petroleum jelly (Vaseline®) to afford a uniform ointment in which the compound retains its stability and oxidation capacity. The compounds of the invention may be formulated into discrete solid forms (such as tablets which disintegrate in water, etc.).

Particularly beneficial applications of the α-amino acid hydrogen peroxide solvates of the invention include their use in cosmetic and personal care products such as hair dyes, tooth pastes, teeth whitening coatings and odor mitigating formulations.

In addition the compounds of the invention may be used as biocidic and/or disinfection agents for bottled water, water reservoirs etc.

The compounds of the invention may also be used for medical and cosmetic skin treatments such as wound disinfection, acne treatment and mouth ulcers.

The compounds of the invention can be used in electrochemical cells (including fuel cells) as an oxidant source.

Formulations containing the compound of the invention can be used for the disinfection or preservation of food including canned food and other food and beverage products.

Formulations containing the above mentioned materials can be used as aquaculture and aquarium additives.

Formulations containing the compounds of the invention can be used as oxidizers in luminescence devices such as disposable luminescence candles, luminescence wires and other light marking devices.

The compounds of the invention can be used in solid phase organic synthesis as an oxidation agent or for radical generation.

Formulations containing the compounds of the invention can be used in bioorganic synthesis as a chiral hydrogen peroxide source.

Formulations containing the compound of the invention can be used in hydrogen peroxide therapy as a source of hydrogen peroxide.

The compounds of the invention can be used as components of abrasive materials for surface treatment of semiconductors, metal oxides, chalcegonides or other materials.

Crystals of the compounds of the invention can be used as optical birefringence materials, as nonlinear optic components and as proton conductors.

The compounds of the invention may also be used for generating anhydrous hydrogen peroxide through a safe and convenient procedure. We have found that an anhydrous (or substantially anhydrous) α-amino acid hydrogen peroxide solvate of the invention, when placed in a suitable organic solvent for a sufficient period of time, undergoes desolvation resulting in hydrogen peroxide molecules being released into the organic solution and essentially solvent-free crystals of the α-amino acid being formed. The de-solvated crystals can be separated from the organic solution, e.g., by filtration, thus leaving an anhydrous hydrogen peroxide reagent (in the form of an organic solution), which is suitable for use as an oxidizer in organic synthesis carried out under anhydrous environment.

Accordingly, the present invention further provides a process for preparing anhydrous hydrogen peroxide, comprising desolvating an anhydrous (or substantially anhydrous) α-amino acid hydrogen peroxide solvate of the invention in an organic solvent and separating the resultant de-solvated α-amino acid crystals from the organic solvent, to obtain anhydrous organic solution of hydrogen peroxide.

An organic solvent suitable for use in the process set out above should not undergo oxidization in the presence of hydrogen peroxide and should also meet the following conditions: i) the solvent dissolves hydrogen peroxide to an appreciable extent and ii) the solvent is a “poor solvent” with respect to the α-amino acid, namely, the α-amino acid is practically insoluble in the solvent. The organic solvent is preferably a poor solvent with respect to the de-solvated crystals of the α-amino acid at room temperature, such that the desolvation process may be conveniently accomplished at room temperature (i.e., between 20° C. and 30° C.) and said desolvated crystals can be easily removed from the solution to give a liquid, anhydrous hydrogen peroxide reagent. Suitable organic solvents may be selected from the group consisting of esters, ketones and ethers. Esters such as ethyl acetate or methyl acetate are preferred. It should be understood that the organic solvent employed in the desolvation process may be a mixture of two or more organic solvents.

The weight ratio between the crystals of the α-amino acid hydrogen peroxide solvate and the organic solvent may be from 0.01 to 50%. In general, the desolvation process lasts between a few hours to a few days. The anhydrous hydrogen peroxide reagent may be analyzed for the presence of residual water and/or α-amino acid using either HPLC, ¹⁷O NMR or ¹H NMR spectroscopy.

The concentration of the hydrogen peroxide in the anhydrous reagent obtained is preferably not less than 0.01% by weight, for example between 1% and 15% by weight. The desired concentration may be adjusted upon partial or complete removal of the organic solvent. Essentially pure hydrogen peroxide can be obtained from the anhydrous hydrogen peroxide organic solution described above by removal of the organic solvent using methods known in the art (e.g., standard evaporation techniques, most conveniently by low-temperature vacuum evaporation). By the term “essentially pure hydrogen peroxide” is meant hydrogen peroxide with a purity level of not less than 90%, and preferably not less than 98% and even more preferably not less than 99%, as determined by permangantometry (i.e. titration by calibrated sodium permanganate solution).

It may be appreciated that when the preparation of pure hydrogen peroxide is contemplated, then the organic solvent employed for the desolvation of the α-amino acid hydrogen peroxide solvate is preferably a volatile solvent, such that it can be easily evaporated from the anhydrous hydrogen peroxide organic solution to afford the essentially pure hydrogen peroxide. Suitable volatile solvents have vapor pressure at the relevant temperature of 0-40° C. which is substantially lower than that of hydrogen peroxide under the same temperatures. Vacuum distillation at room temperature or rotavaporization may be used to accelerate the rate of solvent evaporation.

Accordingly, the present invention further provides a process for preparing essentially pure hydrogen peroxide, comprising desolvating an anhydrous (or substantially anhydrous) α-amino acid hydrogen peroxide solvate of the invention in an organic solvent, separating the resultant de-solvated α-amino acid crystals from said organic solvent, to obtain anhydrous organic solution of hydrogen peroxide, and removing the organic solvent of said solution to form pure hydrogen peroxide.

We have also found that it is possible to crystallize β-amino acids from aqueous hydrogen peroxide solutions, to form n-amino acid hydrogen peroxide solvates in a crystalline, preferably anhydrous, form. The β-amino acid hydrogen peroxide solvates, which form another aspect of the invention, can be prepared using the methods set out above, namely, by dissolving the β-amino acid in a concentrated aqueous solution of hydrogen peroxide, causing a product to precipitate from the solution, and separating the solid formed. The invention thus provides compounds which have the formula β-amino acid.x(H₂O₂).y(H₂O), with x and y being as defined above.

One class of β-amino acids, which can be crystallized and recovered from aqueous solutions of hydrogen peroxide in the form of perhydrates, includes non-polar β-amino acids, having nonpolar side chains as listed above, for example, aliphatic side chains (e.g., straight or branched C1-C5 alkyl chains). Specifically, the invention provides β-alanine dihydrogen peroxide solvate, and a method for obtaining the same, by crystallizing β-alanine from highly concentrated hydrogen peroxide solution (e.g., 70%-98% H₂O₂ aqueous solution), and collecting the crystals formed. The β-amino acid hydrogen peroxide solvates of the invention can be conveniently formulated into useful compositions as described above, and may be used in the applications described above for the α-amino acid hydrogen peroxide solvates.

In the drawings:

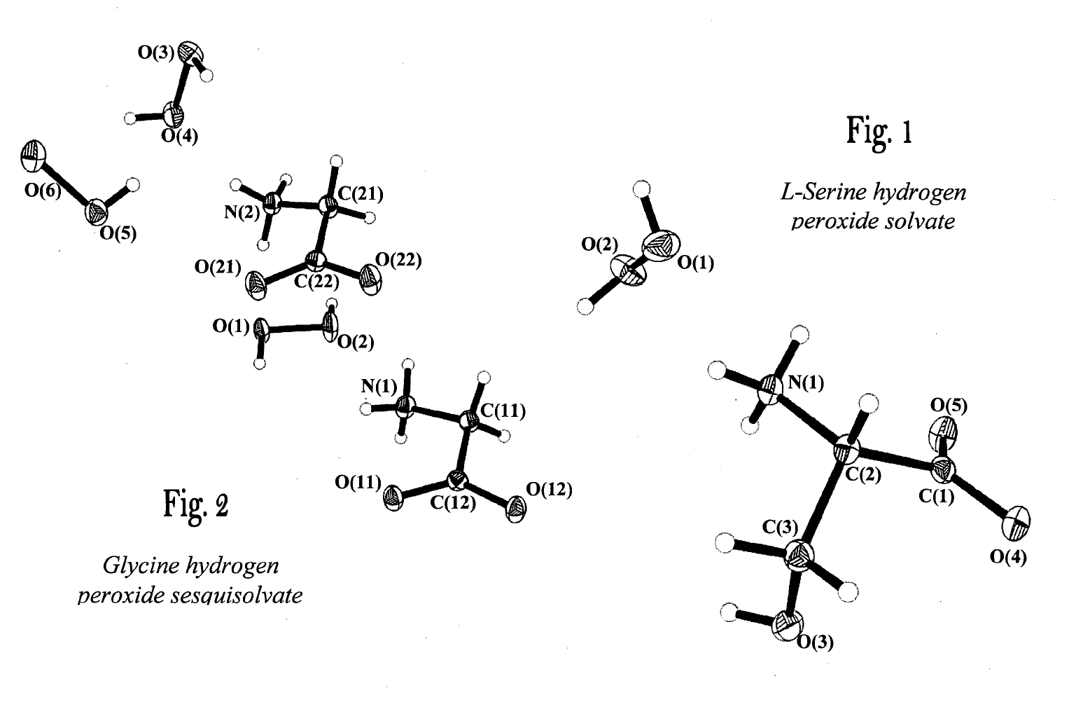

FIG. 1 depicts the configuration of L-Serine hydrogen peroxide solvate as derived from single crystal X-ray crystallography.

FIG. 2 depicts the configuration of Glycine hydrogen peroxide sesquisolvate as derived from single crystal X-ray crystallography.

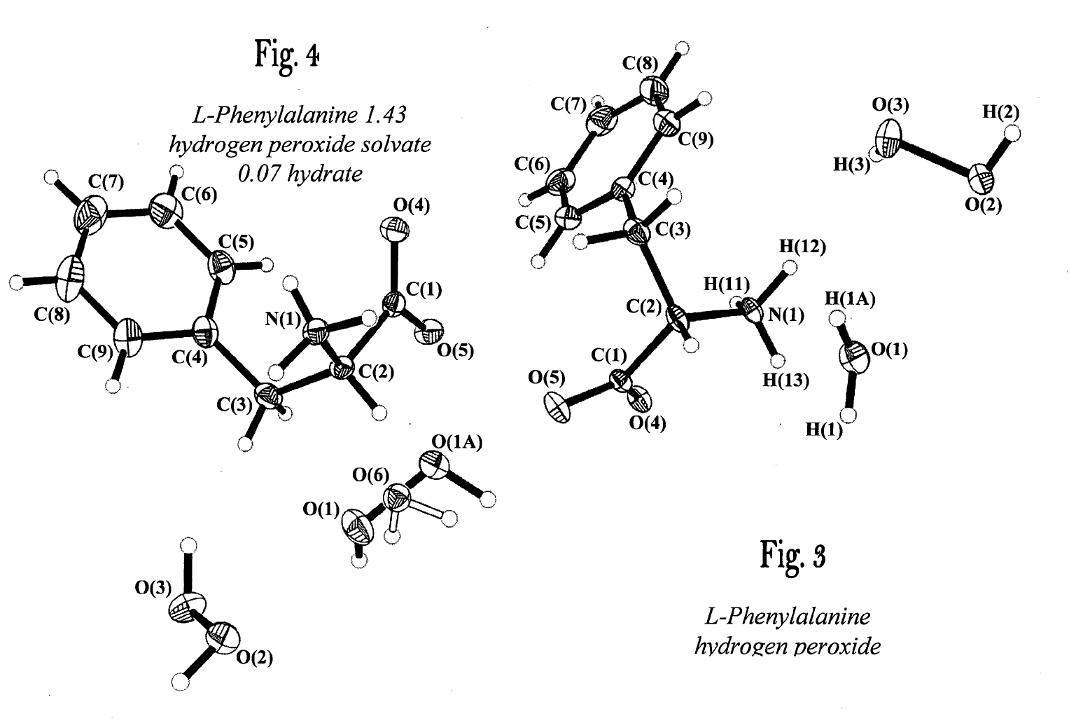

FIG. 3 depicts the configuration of L-Phenylalanine hydrogen peroxide solvate hemihydrate as derived from single crystal X-ray crystallography.

FIG. 4 depicts the configuration of L-Phenylalanine 1.43 hydrogen peroxide solvate 0.07 hydrate as derived from single crystal X-ray crystallography.

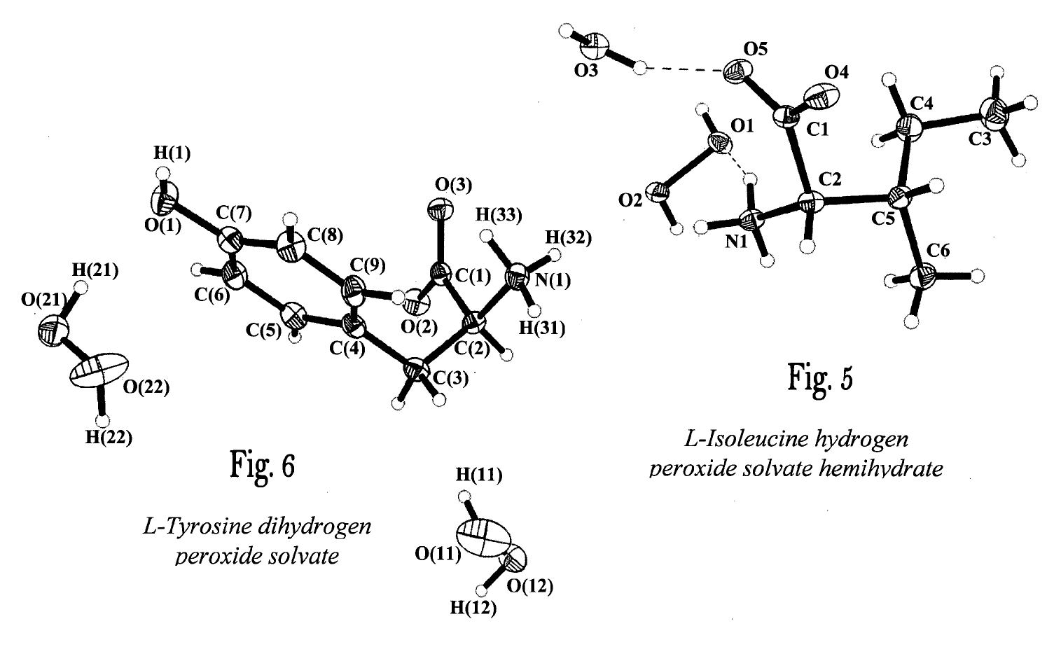

FIG. 5 depicts the configuration of L-Isoleucine hydrogen peroxide solvate hemihydrate as derived from single crystal X-ray crystallography.

FIG. 6 depicts the configuration of L-Tyrosine dihydrogen peroxide solvate as derived from single crystal X-ray crystallography.

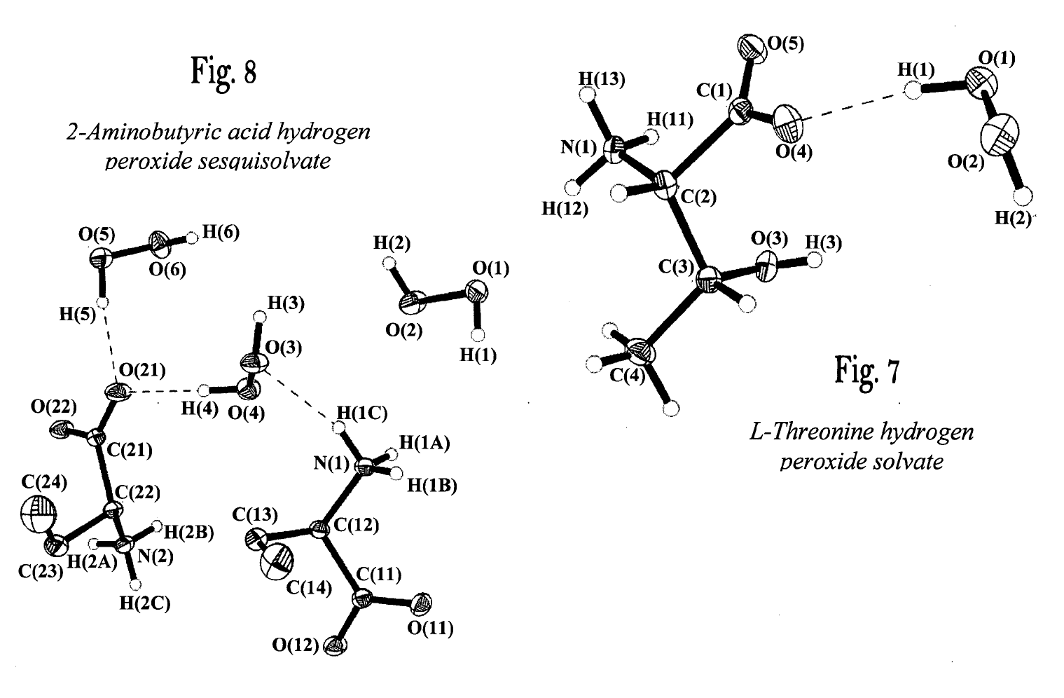

FIG. 7 depicts the configuration of L-Threonine hydrogen peroxide solvate as derived from single crystal X-ray crystallography.

FIG. 8 depicts the configuration of 2-Aminobutyric acid hydrogen peroxide sesquisolvate as derived from single crystal X-ray crystallography.

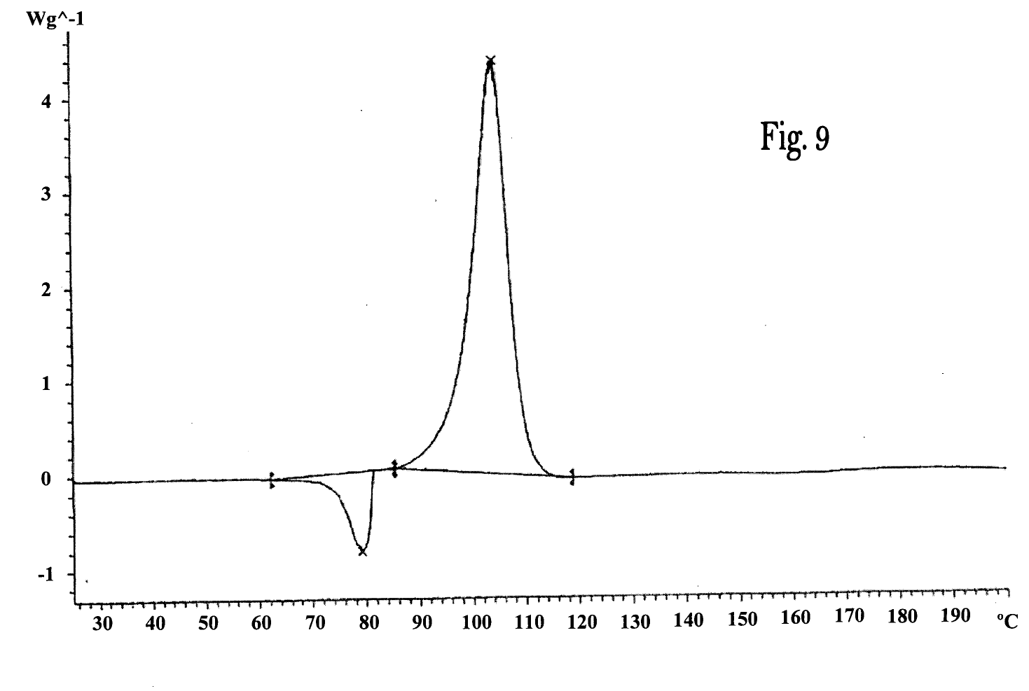

FIG. 9 is a characteristic differential scanning calorimetry thermogram of L-Serine mono hydrogen peroxide solvate.

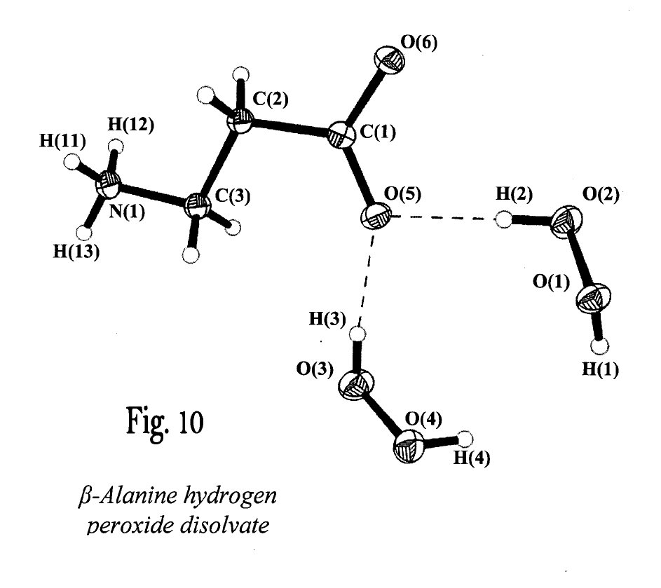

FIG. 10 depicts the configuration of β-alanine dihydrogen peroxide solvate as derived from single crystal X-ray crystallography.

### EXAMPLES

Amino acids were purchased from Sigma-Aldrich. 50% and 70% H₂O₂ solutions were received from Makhteshim Ltd (Beer-Sheva, Israel). 98% H₂O₂ solution was prepared by vacuum distillation of 50% solution (procedures for preparing concentrated peroxide solutions can be found in Schumb, W. C.; Satterfield, C. N.; Wentworth, R. P. Hydrogen peroxide; Reinhold Publishing Corp.: New York, 1955). Organic solvents (ethyl acetate, methyl acetate) were purchased from Sigma Aldrich.

X ray crystallography experimental datasets were collected on a Bruker SMART APEX II diffractometer using graphite monochromatized Mo—Kα radiation (λ=0.71073 Å) at 150 K. The unit cell dimension is defined by three parameters: length of the sides of the cell, relative angles of sides to each other and the volume of the cell. The lengths of the sides of the unit cell are defined by a, b and c. The relative angles of the cell sides are defined by α, β and γ. The volume of the cell is defined as V.

TG and DSC studies were performed on Thermobalance, TG50 and differential scanning calorimeter, DSC 822 (Mettler, Toledo) in the range 25-200° C. under nitrogen flow at a heating rate of 2 degree/min.

FTIR studies were conducted using an Alpha model spectrometer, equipped with a single reflection diamond ATR sampling module, manufactured by Bruker Optik GmbH (Ettlingen, Germany), with 50 scans at 25° C.

¹H and ¹⁷O NMR spectra were collected on a Bruker Avance-500 (11.7483T) spectrometer at resonance frequency of 500.2 and 67.8 MHz, respectively. The measurements were performed using a single pulse sequence with rf pulse duration of 10.9 and 8 μs, and recycling time of 7.34 s and 0.031 s for ¹H and ¹⁷O NMR, respectively. Experiments were carried out at 25° C. The ¹H and ¹⁷O chemical shifts were measured relative to water.

### Example 1

### L-Serine Mono Hydrogen Peroxide Solvate C₃H₇NO₃.H₂O₂

L-serine (20 g) was dissolved in 20 mL of 70% aqueous hydrogen peroxide solution in a round-bottomed flask. The solution was allowed to stand at a temperature of 0° C. for one week, following which crystals were formed. The crystals were separated from the mother liquor by decantation, washed twice with dry ethyl acetate, and dried in a vacuum desiccator for two hours. The crystals were stored in a desiccator under refrigeration. The product obtained was identified as serine mono perhydrate. The yield was 85%. The product has been examined by several techniques as described below.

Crystal data: C₃H₉N₁O₅, M=139.11, orthorhombic, a=4.8616(6), b=9.1187(11), c=13.2712(16) Å, space group P2₁2₁2₁, Z=4, μ(Mo—Kα)=0.151 mm⁻¹, 6747 reflections measured, 860 unique (Rᵢₙₜ=0.0184) which were used in further calculations. The final residuals were: R₁=0.0294, wR₂=0.0777 for 852 reflections with I>2σ(I) and 0.0297, 0.0782 for all data and 106 parameters. The configuration of serine mono perhydrate is shown in FIG. 1.

FTIR: a broad band with a maximum around 3200 cm⁻¹ is attributed to O—H stretching of H₂O₂. A peak at 3500 corresponding to O—H stretching vibrations of the serine hydroxyl group. An additional broad band at 2780 cm⁻¹ is attributed to O—O—H stretching. A characteristic C—H vibration band at 2800 cm⁻¹.

DSC: The differential scanning calorimetry thermogram of serine mono perhydrate is shown in FIG. 9. Serine mono perhydrate exhibits an endothermic melting peak starting at 74° C. with a peak at about 79° C. An exothermic decomposition peak appears above the melting temperature (at about 85° C.) TG indicates that the decomposition event is accompanied by about 25% weight loss.

Melting temperature: 72-74° C. (capillary method).

The serine mono perhydrate crystals were stable and could be stored at room temperature for at least three weeks with no loss of active oxygen.

### Example 2

### L-Serine Perhydrate/Hydrate C₃H₇NO₃.0.91(H₂O₂).0.09(H₂O)

L-serine (1 g) was dissolved in 1 mL of 50% aqueous hydrogen peroxide solution in a round-bottomed flask. The solution was allowed to stand at a temperature of −20° C. for 5 hours, following which crystals were formed. The crystals were separated from the solution according to the procedure of Example 1.

The crystals were analyzed by single crystal x-ray, elemental analysis, hydrogen peroxide content, (by permanganatometry) and x-ray powder diffraction to assess the hydrogen peroxide content. The product was identified as L-Serine perhydrate (L-serine 0.91 hydrogen peroxide solvate 0.09 hydrate C₃H₇NO₃ 0.91 (H₂O₂) 0.09 (H₂O)).

### Example 3

### Glycine Hydrogen Peroxide Sesquisolvate C₂H₅NO₂.1.5(H₂O₂)

Glycine (0.8 g) was dissolved in 1 mL of 98% aqueous hydrogen peroxide solution in a round-bottomed flask. The solution was allowed to stand at a temperature of −20° C. for a week, following which crystals were formed. The crystals were separated from the solution according to the procedure of Example 1. The product obtained was identified as glycine hydrogen peroxide sesquisolvate, with hydrogen peroxide content of about 40.47%. The yield was 85%. The compound has been examined by the methods described below.

Crystal data: C₄H₁₆N₂O₁₀, M=252.19, triclinic, a=7.2854(4), b=8.0045(5), c=9.6698(6) Å, α=79.485(1), β=72.238(1), γ=87.275(1)°, V=527.99(5) Å³, space group P-1, Z=2, μ(Mo—Kα)=0.159 mm⁻¹, 5439 reflections measured, 2543 unique (Rᵢₙₜ=0.0126) which were used in further calculations. The final residuals were: R₁=0.0291, wR₂=0.0812 for 2334 reflections with I>2σ(I) and 0.0315, 0.0828 for all data and 209 parameters. The configuration of glycine hydrogen peroxide sesquisolvate is shown in FIG. 2.

FTIR: a broad band with a maximum around 3200 cm⁻¹ is attributed to O—H stretching of H₂O₂. Strong bands of NH stretching occur in the same region.

DSC: The DSC curve exhibits an endothermic melting peak starting at 60° C. with a peak at 63° C. and an exothermic decomposition peak starting at 80° C. (accompanied by 42% weight loss, as determined by TG).

Melting temperature: 56-60° C. (capillary method).

### Example 4

### L-Phenylalanine Hydrogen Peroxide Solvate Hemihydrate C₉H₁₁NO₂.H₂O₂.0.5 (H₂O)

L-phenylalanine (0.2 g) was dissolved in 1 mL of 50% aqueous hydrogen peroxide solution in a round-bottomed flask. The solution was allowed to stand at a temperature of 0° C. for a several days, following which crystals were formed. The crystals were separated from the solution according to the procedure of Example 1. The crystals were identified as the entitled product. The yield was 70%. The product was examined by single crystal X-ray.

Crystal data: C₁₈H₂₈N₂O₉, M=416.42, Monoclinic, a=10.0268(16), b=7.2283(12)), c=14.150(2) Å, α=90°, β=92.780 (3)°, γ=90°, V=1024.3(3) Å³, space group C2, Z=2, μ(Mo—Kα)=0.109 mm⁻¹, 3278 reflections measured, 1307 unique (Rᵢₙₜ=0.0251) which were used in further calculations. The final residuals were: R₁=0.0315, wR₂=0.0788 for 1205 reflections with I>2σ(I) and 0.0364, 0.0809 for all data and 156 parameters. The configuration of phenylalanine hydrogen peroxide solvate hemihydrate is shown in FIG. 3.

### Example 5

### L-Phenylalanine Hydrogen Peroxide Solvate Hydrate C₉H₁₁NO₂.1.43(H₂O₂).0.07(H₂O)

L-phenylalanine (1 g) was dissolved in 1 mL of 98% aqueous hydrogen peroxide solution in a round-bottomed flask. The solution was allowed to stand at a temperature of 0° C. for one week, following which crystals were formed. The crystals were separated from the solution according to the procedure of Example 1. The crystals were identified by single crystal x-ray as the entitled product.

Crystal data: C₉H₁₄NO₄, M=215.09, Monoclinic, a=10.2375(8), b=7.2175(5), c=14.0450(11) Å, α=90°, β=92.2770 (10)°, γ=90°, V=1036.95(14) Å³, space group C2, Z=4, μ(Mo—Kα)=0.113 mm⁻¹, 5627 reflections measured, 1354 unique (Rᵢₙₜ=0.0177) which were used in further calculations. The final residuals were: R₁=0.0283, wR₂=0.0744 for 1326 reflections with I>2σ(I) and 0.0290, 0.0749 for all data and 162 parameters. The configuration of phenylalanine hydrogen peroxide solvate hemihydrate is shown in FIG. 4.

### Example 6

### L-Isoleucine Hydrogen Peroxide Solvate Hemihydrate C₆H₁₃NO₂.H₂O₂.0.5(H₂O)

L-isoleucine (0.25 g) was dissolved in 1 mL of 50% aqueous hydrogen peroxide solution in a round-bottomed flask. The solution was allowed to stand at a temperature of 0° C. for a week following which crystals were formed. The crystals were separated from the solution according to the procedure of Example 1. The crystals were identified as the entitled product. The yield was 80%.

Crystal data: C₁₂H₃₂N₂O₉, M=348.4, Monoclinic, a=10.0728(7), b=7.3940(6)), c=12.1940(7) Å, α=90°, β=91.839 (3)°, γ=90°, V=907.72(11) Å³, space group C2, Z=2, μ(Mo—Kα)=0.108 mm⁻¹, 3592 reflections measured, 1237 unique (Rᵢₙₜ=0.0270) which were used in further calculations. The final residuals were: R₁=0.0295, wR₂=0.0671 for 1123 reflections with I>2σ(I) and 0.0351, 0.0691 for all data and 131 parameters. The configuration of L-isoleucine hydrogen peroxide solvate hemihydrate is shown in FIG. 5.

### Example 7

### L-Tyrosine Dihydrogen Peroxide Solvate C₉H₁₁NO₃.2(H₂O₂)

L-tyrosine (0.15 g) was dissolved in 1 mL of 50% aqueous hydrogen peroxide solution in a round-bottomed flask. The solution was allowed to stand at a temperature of 0° C. for a week, following which crystals were formed. The crystals were separated from the solution according to the procedure of Example 1. The crystals were identified as the entitled product. The yield was 60%.

Crystal data: C₉H₁₅NO₇, M=249.22, Monoclinic, a=8.7005(19), b=5.9331(13), c=10.929(2) Å, α=90°, β=94.650(4)°, γ=90°, V=562.3(2) Å³, space group P2₁, Z=2, μ(Mo—Kα)=0.128 mm⁻¹, 31716 reflections measured, 1326 unique (Rᵢₙₜ=0.0395) which were used in further calculations. The final residuals were: R₁=0.0419, wR₂=0.0951 for 1191 reflections with I>2σ(I) and 0.0481, 0.0982 for all data and 180 parameters. The configuration of L-tyrosine dihydrogen peroxide solvate is shown in FIG. 6.

### Example 8

### L-Threonine Hydrogen Peroxide Solvate C₄H₉NO₃.H₂O₂

L-threonine (0.9 g) was dissolved in 1 mL of 70% aqueous hydrogen peroxide solution in a round-bottomed flask. The solution was allowed to stand at a temperature of 0° C. for a week, following which crystals were formed. The crystals were separated from the solution according to the procedure of Example 1. The crystals were identified as the entitled product. The yield was 70%.

Crystal data: C₄H₁₁NO₅, M=153.14, orthorhombic, a=6.129(3), b=6.387(3)), c=17.177 (7) Å, α=90°, β=90° (3), γ=90°, V=672.4(3) Å³, space group P2₁P2₁P2₁, Z=4, μ(Mo—Kα)=0.140 mm⁻¹, 7776 reflections measured, 971 unique (Rᵢₙₜ=0.0299) which were used in further calculations. The final residuals were: R₁=0.0372, wR₂=0.0920 for 942 reflections with I>2σ(I) and 0.0382, 0.0928 for all data and 116 parameters. The configuration of L-threonine hydrogen peroxide solvate is shown in FIG. 7.

### Example 9

### L-Norleucine Hydrogen Peroxide Sesquisolvate C₆H₁₃NO₂.1.5H₂O₂

L-norleucine (1 g) was dissolved in 1 mL of 98% aqueous hydrogen peroxide solution in a round-bottomed flask. The solution was allowed to stand at a temperature of 0° C. for a week, following which crystals were formed. The crystals were separated from the solution according to the procedure of Example 1. The crystals were identified as the entitled product by x-ray analysis. The yield was 75%.

### Example 10

### 2-Aminobutyric Acid Hydrogen Peroxide Sesquisolvate C₄H₉NO₂.1.5(H₂O₂)

2-aminobutyric acid (0.6 g) was dissolved in 1 mL of 98% aqueous hydrogen peroxide solution in a round-bottomed flask. The solution was allowed to stand at a temperature of 0° C. for one week, following which crystals were formed. The crystals were separated from the solution according to the procedure of Example 1. The crystals were identified as the entitled product. The yield was 60%.

Crystal data: C₈H₂₄N₂O₁₀, M=308.29, Triclinic, a=8.0479(14), b=9.6064(17), c=9.7464(17) Å, α=81.813(3)°, β=81.415(2)°, γ=88.676(3)°, V=737.5(2) Å³, space group P-1, Z=2, μ(Mo—Kα)=0.128 mm⁻¹, 7086 reflections measured, 3224 unique (Rᵢₙₜ=0.0229) which were used in further calculations. The final residuals were: R₁=0.0349, wR₂=0.0908 for 2805 reflections with I>2σ(I) and 0.0404, 0.0940 for all data and 231 parameters. The configuration of 2-aminobutyric acid hydrogen peroxide sesquisolvate is shown in FIG. 8.

### Example 11

### L-Valine Hydrogen Peroxide Solvate

L-valine (1 g) was dissolved in 1 mL of 98% aqueous hydrogen peroxide solution in a round-bottomed flask. The solution was allowed to stand at a temperature of 0° C. for a week, following which crystals were formed. The crystals were separated from the solution according to the procedure of Example 1.

### Example 12

### Preparation of Anhydrous Hydrogen Peroxide

The following example illustrates the utility of the amino acid perhydrates of the invention in the preparation of anhydrous hydrogen peroxide.

1 g of L-Serine mono hydrogen peroxide solvate (the product of Example 1) was added to 10 ml of ethyl acetate placed in a beaker, which was left unstirred for two days. The crystals were then separated from the liquid phase by filtration using glass filter (Whatman). The crystals and the filtrate were subjected to analysis indicating that desolvation of the serine perhydrate took place, generating serine crystals accompanied by the release of hydrogen peroxide into the organic solvent. The details of the analysis are as follows.

### Analysis of the Crystals:

The powder X-ray diffraction pattern of the crystals obtained agrees well with the reported x-ray powder diffraction pattern of serine.

### Analysis of the Filtrate:

¹H NMR of the same anhydrous solution show the signals of the ethyl acetate (1.6, 2.4 and 4.5 ppm) and a singlet of hydrogen peroxide at app. 9.8 ppm. Water signal was completely absent in the anhydrous solution. The recovery of the hydrogen peroxide was quantitative as determined by permanganatometry. The residual amount of serine in the anhydrous hydrogen peroxide solution was determined by immersing of 1 gr of the perhydrate in 1 to 20 ml of methyl or ethyl acetate solvents. In all cases the concentration of serine was less than our limit of quantification by HPLC, 0.4 mM (which corresponds to serine/hydrogen peroxide ratio<0.003).

### Example 13

### Preparation of Pure Hydrogen Peroxide

The procedure described in Example 12 was repeated, using methyl acetate as the organic solvent in place of ethyl acetate. Following the separation of the serine crystals, the filtrate, consisting of an anhydrous solution of hydrogen peroxide in methyl acetate, was concentrated in order to recover pure hydrogen peroxide. To this end, the solution was rotavapored under vacuum, using oil pump and liquid nitrogen trap. High purity hydrogen peroxide was obtained (99.4%), with hydrogen peroxide yield of 60%.

### Example 14

### A Cosmetic Composition Containing Serine Perhydrate Crystals

50 mg serine mono perhydrate crystals prepared according to Example 1 were crushed in a mortar and were mixed with 400 mg Vaseline® (pharmaceutical grade) to form homogenous mixture. The resulting ointment was kept in a closed beaker at room temperature.

After seven days, the ointment formulation was visually checked, and it was found that it retained its homogeneity and appearance. The hydrogen peroxide content of the cream formulation was measured using permanganate and iodine titration, which showed no reduction of active oxygen content during the seven days storage period.

### Example 15

### β-Alanine Hydrogen Peroxide Disolvate C₃H₇NO₂.2(H₂O₂)

β-Alanine (1 g) was dissolved in 1 mL of 98% aqueous hydrogen peroxide solution in a round-bottomed flask. The solution was allowed to stand at a temperature of 0° C. for several days, following which crystals were formed. The crystals were separated from the solution according to the procedure of Example 1. The crystals were identified as the entitled product. The yield was 70%. The product was examined by single crystal X-ray.

Crystal data: C₃H₁₁N₁O₆, M=157.13, monoclinic, a=6.8875(11), b=9.5679(16), c=10.5760(18), α=90°, β=101.842(2)°, γ=90° Å, space group P2₁/n, Z=4, μ(Mo—Kα)=0.150 mm⁻¹, 6816 reflections measured, 1638 unique (Rᵢₙₜ=0.0218) which were used in further calculations. The final residuals were: R₁=0.0285, wR₂=0.0759 for 1514 reflections with I>2σ(I) and 0.0309, 0.0772 for all data and 135 parameters. The configuration of β-Alanine hydrogen peroxide disolvate is shown in FIG. 10.

1-21. (canceled)

22. A compound which is an α-amino acid hydrogen peroxide solvate, wherein the side chain of the α-amino acid has no basic nitrogen.

23. The compound of claim 22, wherein the side chain of the α-amino acid is nonpolar.

24. The compound of claim 23, wherein the α-amino acid is selected from the group consisting of glycine, valine, leucine, isoleucine, norleucine, 2-aminobutyric acid and phenylalanine.

25. The compound of claim 22, wherein the side chain of the α-amino acid contains one or more polar functionalities.

26. The compound of claim 25, where the polar functionality is a hydroxy group.

27. The compound of claim 26, wherein the α-amino acid is selected from the group consisting of serine, threonine and tyrosine.

28. The compound of claim 22 having the formula:
α-amino acid.x(H₂O₂).y(H₂O)
wherein the side chain of said α-amino acid is selected from the group consisting of:
(i) the side chains of proteinogenic α-amino acids, excluding the basic proteinogenic α-amino acids;
(ii) structural isomers or homologs of proteinogenic α-amino acid side chains, when said side chains are alkyl groups; and
(iii) hydroxy-substituted derivative of proteinogenic α-amino acid side chains;
the coefficient x is between ⅓ and 4; and
the coefficient y is between 0 and 1.

29. A compound of claim 28, wherein x is between ½ to 2 and y is between 0 and 0.5.

30. A compound according to claim 22, which is anhydrous.

31. The anhydrous compound according to claim 30, wherein x is 1.5 or 2.

32. A compound according to claim 22, characterized in that its DSC curve exhibits a melting endotherm followed by a distinct exothermic peak.

33. A compound according to claim 22, having hydrogen peroxide content of not less than 18% by weight.

34. A compound according to claim 28, which is selected from the group consisting of:
L-Serine hydrogen peroxide solvate [C₃H₇NO₃.H₂O₂)];
L-Tyrosine dihydrogen peroxide solvate [C₉H₁₁NO₃.2(H₂O₂)];
Glycine hydrogen peroxide sesquisolvate [C₂H₅NO₂O.1.5(H₂O₂)];
L-Phenylalanine hydrogen peroxide solvate hemihydrate [C₉H₁₁NO₂.H₂O₂.0.5(H₂O)];
L-Serine 0.91 hydrogen peroxide solvate 0.09 hydrate [C₃H₇NO₃.0.91(H₂O₂).0.09(H₂O)];
L-Phenylalanine 1.43 hydrogen peroxide solvate 0.07 hydrate [C₉H₁₁NO₂.1.43(H₂O₂).0.07(H₂O)];
L-Isoleucine hydrogen peroxide solvate hemihydrate [C₆H₁₃NO₂.H₂O₂.0.5(H₂O)];
L-Threonine hydrogen peroxide solvate [C₄H₉NO₃.H₂O₂];
L-Norleucine hydrogen peroxide sesquisolvate [C₆H₁₃NO₂.1.5H₂O₂];
2-Aminobutyric acid hydrogen peroxide sesquisolvate [C₄H₉NO₂.1.5(H₂O₂)]; and
L-Valine hydrogen peroxide solvate.

35. A process for preparing the α-amino acid hydrogen peroxide solvate as defined in claim 22, comprising the steps of (a) providing a solution of hydrogen peroxide, (b) contacting the solution with an α-amino acid, wherein the side chain of the α-amino acid has no basic nitrogen and (c) precipitating the α-amino acid hydrogen peroxide solvate and separating the same from the solution.

36. A process according to claim 35, wherein the hydrogen peroxide solution is an aqueous solution having concentration of not less than 50%.

37. A process according to claim 36, wherein the hydrogen peroxide solution is an aqueous solution having concentration of not less than 70%, and the α-amino acid hydrogen peroxide solvate prepared is anhydrous.

38. A composition comprising one or more α-amino acid hydrogen peroxide solvates as defined in claim 22 and at least one carrier.

39. A process, comprising desolvating an anhydrous α-amino acid hydrogen peroxide solvate as defined in claim 22 in an organic solvent, and separating the resultant de-solvated α-amino acid crystals from the organic solvent, to obtain anhydrous organic solution of hydrogen peroxide.

40. A process according to claim 39, which further comprises the step of removing the organic solvent from the anhydrous organic solution of hydrogen peroxide to form essentially pure hydrogen peroxide.

41. β-amino acid hydrogen peroxide solvate.

42. The compound of claim 41, which is crystalline β-Alanine dihydrogen peroxide solvate having the formula C₃H₇NO₂.2(H₂O₂).
